(19) United States
(12) Patent Application Publication
(10) Pub. No.: US 2008/0177068 A1
(43) Pub. Date: Jul. 24, 2008

(54) QUINAZOLE DERIVATIVES

(76) Inventors: Wenlin Huang, Guangzhou City (CN); Xiaohong Zhou, Guangzhou City (CN)

Correspondence Address: OCCHIUTI ROHLICEK & TSAO, LLP, 10 FAWCETT STREET, CAMBRIDGE, MA, 02138, US

(21) Appl. No.: 11/939,244

(22) Filed: Nov. 13, 2007

Related U.S. Application Data
(63) Continuation-in-part of application No. PCT/CN2005/000663, filed on May 12, 2005.

(51) Int. Cl. C07D 239/94

(52) U.S. Cl. 544/293

(57) ABSTRACT

The present invention relates to quinazoline derivatives represented by general formula (I):

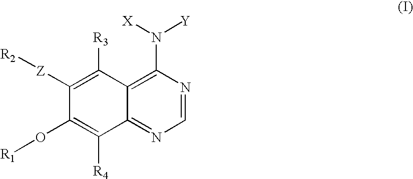

wherein X, Y, Z, R₁, R₂, R₃, and R₄ are as defined herein. The invention also relates to a method of preparing these compounds, and use of these compounds for inhibiting tumor growth.

### CROSS REFERENCE

This application is a continuation-in-part of PCT Application No. PCT/CN2005/000663, filed on May 12, 2005. The contents of the international application are incorporated by reference.

### BACKGROUND

Solid tumors rely on tumor neovascularization in their formation, development, recurrence and metastasis. In other words, vascularization of a tumor is a prerequisite for the growth and metastasis of solid tumors. Hunger therapy of tumor, namely inhibition of tumor neovascularization by cutting off the blood supply to tumor tissue, is deemed as one of the most promising new methods of treatment of solid tumors.

Formation of normal tissue and maintenance of its function rely on the transmembrane signaling cascade from the cytoplasm to the nucleus, which controls the transcription and regulation of genes. Cancer is the result of abnormal cellular activities, such as changes in cell growth, cell survival, and cellular function, and loss of differentiation ability of cell caused by disordered signaling pathway to form a tumor. The development of tumor relies on its host by way of neovascularization to utilize the nutrient and oxygen in the host, during which certain growth factor from tumor stimulates the signaling of host endothelial cell and promotes tumor angiogenesis by extending the existing vessels. The angiogenesis rate in adults is very low, and only the endometrium retains normal angiogenesis activity. For the above reasons, it will be very effective to block the formation of pathogenic vessels by targeting the signaling pathway involved in angiogenesis.

Vascular Endothelial Growth Factor (VEGF) is a growth factor involved in tumor angiogenesis, and plays a role in hormonal regulation of the differentiation of endothelial cell. Solid tumor development is closely related with expression of VEGF. It is shown by current studies that many diseases, including malignant tumors, are associated with angiogenesis (Fan, et al, 1995, Trends Pharmacol. Sci. 16, 57-66; Folkman, 1995, Nature Medicine, 27-31). Change in vasopermeability is thought to have a role in both normal and pathological physiological processes (Cullinan-Bove, et al, 1993, Endocrinology 133, 829-837; Senger, et al, 1993, Cancer and Metastasis Reviews. 12, 303-324), and VEGF is an important stimulating factor in the normal and pathological angiogenesis and change in vasopermeability (Jakeman, et al, 1993, Endocrinology 133, 848-859; Kolch, et al, 1995, Breast Cancer Research and Treatment, 36, 139-155; Connolly, et al, 1989, J. Biol. Chem. 264, 20017-20024). Tumor growth can be inhibited by VEGF antagonism by use of sequestration of VEGF by antibody (Kim, 1993, Nature 362, 841-844).

Improved expression of VEGF is caused by stimulation of multiple factors, such as activation of proto-oncogene and hypoxemia. Hypoxemia of solid tumor may result from the improper perfusion of tumor patient. Besides promotion of neovascularization, VEGF has effect in improvement of vasopermeability, which accelerates the exchange of nutrient and metabolite between tumor and neighboring tissue, and overcomes the barricade of vessel wall to allow metastasis of tumor to distant tissues.

VEGF has tyrosine kinase activity, and can activate the related signaling pathway and promote the tumor neovascularization upon binding with tyrosine kinase as its receptor. Receptor tyrosine kinases (RTKs) activated by the binding of VEGF and its receptor plays an important role in the biochemical signal transduction across cytoplasmic membrane, and can influence growth and metastasis of tumor. The trans-membrane molecule is characterized by that the extracellular ligand binding domain and endocellular tyrosine kinase domain are linked by the fragment in the cytoplasmic membrane. The binding of ligand and receptor stimulates the tyrosine kinase activity associated with receptor, and induces the phosphorylation of tyrosine residue on the receptor and other endocellular molecules. The phosphorylation of tyrosine switches on the signal cascade, and produces multiple cellular responses. Till now at least 19 different RTK subfamilies defined by amino acid sequence homology have been identified, one of which includes Flt (also called Flt1) and Flt4 (both of which are similar to fms), and KDR (also called Flk-1) with kinase domain region. It has been proved that two of the relevant RTKs, Flt and KDR, can bind with VEGF with high affinity (De Vries, et al, 1992, Science 255, 989-991; Terman, et al, 1992, Boichem. Biophys. Res. Comm., 1992, 187, 1579-1586). Binding of VEGF with receptor expressed in heterogenous cell is related with tyrosine phosphorylation level of protein and change in calcium flow.

It is proved by the above mentioned studies that VEGF is specific to neovascularization of solid tumor and is a critical regulating factor directly and positively regulating vascular endothelial cells. VEGF-KDR/Flk-1 pathway becomes one of the major targets in tumor therapy by inhibition of tumor angiogenesis. Inhibition of tyrosine kinase activity is an important way of blocking tumor angiogenesis.

### SUMMARY

The present invention relates to a method of preparing a quinazoline compound of formula (I):

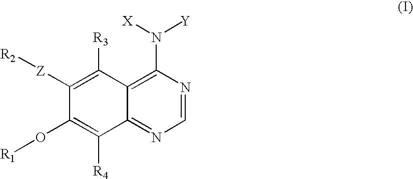

wherein X represents H or C₁-C₄ alkyl, preferably H or methyl, and more preferably H; Y represents

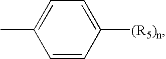

in which n is 1, 2, 3, or 4, and R₅, being the same or different groups, is H, methyl, trifluoromethyl, nitro, cyano, C₂-C₄ alkyl, C₂-C₄ alkoxyl, N—(C₂-C₄)alkylamine, enzyme, hydroxyl, NN-triaza(C₁-C₄) alkylamine, C₂-C₄ alkylthio or C₂-C₄ alkylsulfonyl; Z is

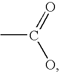

—O, —S or —NH; R₁ represents C₁-C₄ alkyl (e.g., methyl); R₂ represents C₁-C₅ alkyl-R₆, C₂-C₆ alkenyl-R₆, or C₂-C₆ alkynyl-R₆, in which the alkyl, alkenyl, and alkynyl are optionally substituted with one or more alkynyl, enzyme or amino; and R₆ is piperidin-4-yl, optionally substituted with one or more substituents of alkynyl, enzyme or amino; and R₃ and R₄ represent H, C₁-C₄ alkyl, C₂-C₆ alkenyl, C₂-C₆ alkynyl, cycloalkyl, or heterocycloalkyl. The method includes reacting a compound of formula (III) with a compound of formula (IV):

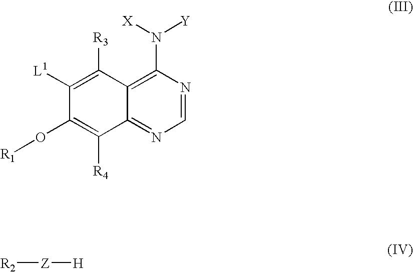

In formula (III) or (IV), L¹ represents a leaving group (such as halogen, alkoxyl, aryloxy, or sulfonoxy, preferably, halogen, e.g., Br, or C₁-C₄ alkoxyl, e.g., methoxy) that can be substituted by another functional group; R₁ represents C₁-C₄ alkyl, preferably methyl; R₃ and R₄, independently, represent H, C₁-C₄ alkyl, C₂-C₆ alkenyl, C₂-C₆ alkynyl, cycloalkyl, or isocycloalkyl, preferably, R₃ is C₁-C₄ alkyl and R₄ is H; X represents H, methyl, or C₁-C₄ alkyl, preferably H; and Y represents

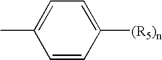

(e.g., methylphenyl), wherein n is 1, 2, 3, or 4, and R₅, being the same or different groups, is H, methyl, trifluoromethyl, nitro, cyano, C₂-C₄ alkyl, C₂-C₄ alkoxyl, N—(C₂-C₄)alkylamine, enzyme, hydroxyl, NN-triaza(C₁-C₄) alkylamine, C₂-C₄ alkylthio, or C₂-C₄ alkylsulfonyl; R₂ represents C₁-C₅ alkyl-R₆, C₂-C₆ alkenyl-R₆, C₂-C₆ alkynyl-R₆, preferably, C₁-C₅ alkyl-R₆ (e.g., 2-piperidin-4-ylethyl); in which the alkyl, alkenyl, and alkynyl are optionally substituted with one or more alkynyl, enzyme or amino; and R₆ is piperidin-4-yl, optionally substituted with one or more substituents of alkynyl, enzyme or amino; and Z represents —O, —NH,

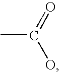

or —S, preferably

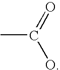

The term “alkyl” refers to a saturated, linear or branched hydrocarbon moiety, such as —CH₃, —CH(CH₃)₂, or —CH₂—. The term “alkenyl” refers to a linear or branched hydrocarbon moiety that contains at least one double bond, such as —CH═CH—CH₃ or —CH═CH—CH₂—. The term “alkynyl” refers to a linear or branched hydrocarbon moiety that contains at least one triple bond, such as —C≡C—CH₃ or —C≡C—CH₂—. The term “cycloalkyl” refers to a C₃-C₈ saturated, cyclic hydrocarbon moiety, such as cyclohexyl or cyclohexylene. The term “heterocycloalkyl” refers to a C₁-C₈ saturated, cyclic moiety having at least one ring heteroatom (e.g., N, O, or S), such as 4-tetrahydropyranyl or 4-tetrahydropyranylene. The term “alkoxyl” refers to a radical of —O-alkyl. The term “alkylamino” refers to an alkyl-substituted amino group. The term “alkylthio” refers to a radical of —S-alkyl.

Alkyl, alkenyl, alkynyl, cycloalkyl, and heterocycloalkyl, mentioned herein include both substituted and unsubstituted moieties, unless specified otherwise. Possible substituents on cycloalkyl and heterocycloalkyl include, but are not limited to, C₁-C₁₀ alkyl, C₂-C₁₀ alkenyl, C₂-C₁₀ alkynyl, C₃-C₂₀ cycloalkyl, C₃-C₂₀ cycloalkenyl, C₁-C₂₀ heterocycloalkyl, C₁-C₂₀ heterocycloalkenyl, C₁-C₁₀ alkoxy, aryl, aryloxy, heteroaryl, heteroaryloxy, amino, C₁-C₁₀ alkylamino, C₁-C₂₀ dialkylamino, arylamino, diarylamino, hydroxyl, halo, thio, C₁-C₁₀ alkylthio, arylthio, C₁-C₁₀ alkylsulfonyl, arylsulfonyl, acylamino, aminoacyl, aminothioacyl, amidino, guanidine, ureido, cyano, nitro, acyl, thioacyl, acyloxy, carboxyl, and carboxylic ester. On the other hand, possible substituents on alkyl, alkenyl, or alkynyl include all of the above-recited substituents except C₁-C₁₀ alkyl.

The term “enzyme” refers to an active protein, which, when attached to the substrate, generates effective physiological reactions.

The compounds described above include the free base compounds, as well as their salts. The salts, for example, can be formed between a positively charged moiety (e.g., amine) on the compounds and an anion. Suitable anions include, but are not limited to, chloride, bromide, iodide, sulfate, nitrate, phosphate, citrate, methanesulfonate, trifluoroacetate, and acetate.

As the compound of formula (I) have inhibitory effect against activities of tyrosine kinases, they can be used to treat tumor. This, this invention relates to a method of treating tumor using the compounds. It also relates to a method of inhibiting tumor growth, VEGF-induced propagation of human umbilical vein endothelial cells, and the activity of KDR tyrosine kinase, using the compound of formula (I).

Also within the scope of this invention is use quinazoline compound to prepare an antiphlogistics or to prepare a medicament for inhibiting tumor growth, VEGF-induced propagation of human umbilical vein endothelial cells, or activity of KDR tyrosine kinase.

Other features or advantages of the present invention will be apparent from the following detailed description of several embodiments and the figures, and also from the appending claims.

### DESCRIPTION OF THE FIGURES

FIG. 1 Dose dependence of epiphyseal growth plate of rat joint on the inventive compound.

FIG. 2 Inhibitive effect of the inventive compound on human prostate tumor transplanted in nude mouse by use of PC-3.

FIG. 3 Inhibitive effect of the inventive compound on the growth of colon carcinoma cell Lovo.

FIG. 4 Inhibitive effect of the inventive compound on transplanted tumor in the nude mouse model constructed by use of colon carcinoma cell LoVo.

Detailed description of the present invention is provided below with the combination of the figures and embodiments.

### DETAILED DESCRIPTION

The above-described reaction can be performed in the presence of base, in which the base is organic amine, carbonate or hydroxide of alkali metal or alkaline earth metal, alkali hydride, amide of alkali metal or alkaline earth metal. Examples of base include, but are not limited to, 2,6-dimethylpyridine, trimethylpyridine, 4-dimethylaminopyridine, triethylamine, morphine, N-methylmorphine, 1,8-diazabicyclo(5,4,0)undecene-7, sodium carbonate, potassium carbonate, calcium carbonate, sodium hydroxide, potassium hydroxide, sodium hydride, sodium amide, or sodium bis(trimethylsilyl)amide.

The above-described reaction can also be performed in the presence of in the presence of an inert solvent. Examples of inert solvents, include, but are not limited to, methanol, ethanol, isopropanol, ethyl acetate, dichloromethane, trichloromethane, carbon tetrachloride, tetrahydrofuran, 1,4-dioxane, toluene, N,N-dimethylformamide, N,N-dimethylacetamide, N-methylpyrrol-2-one, and dimethyl sulfoxide.

In addition, the above-described reaction can also performed at a temperature of 10-150° C., preferably, 100° C.

### Synthesis of Intermediate

A compound of formula (III), used in the above-described method, can be obtained by reducing a nitro compound of formula (V).

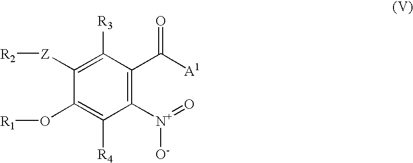

In formula (V):

R₁ represents C₁-C₄ alkyl;

R₂ represents C₁-C₅ alkyl-R₆, C₂-C₆ alkenyl-R₆, or C₂-C₆ alkynyl-R₆, in which the alkyl, alkenyl, and alkynyl are optionally substituted with one or more alkynyl, enzyme or amino; and R₆ is piperidin-4-yl, optionally substituted with one or more substituents of alkynyl, enzyme or amino;

R₃ and R₄ represent —H, C₁-C₄ alkyl, C₂-C₆ alkenyl, C₂-C₆ alkynyl, cycloalkyl, or heterocycloalkyl;

Z represents —O, —NH,

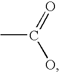

### or —S; and

A¹ represents hydroxyl, alkoxyl (preferably C₁-C₄ alkoxyl), or amino.

Conventional reducing agents can be used in the reaction. As an example, an activated metal, such as iron obtained by washing iron powder with dilute acid (e.g. dilute hydrochloric acid), is used in this reaction. The reduction reaction may be performed under various conditions. For example, a catalytic metal (e.g. Pd or Pt) can be used to catalyze the reaction. The reduction reaction may be promoted by heating solvent/diluent (e.g. mixture of water and alcohol such as methanol or ethanol) carrying nitro compound or activated metal to certain temperature range (e.g. 50-150° C., preferably about 70° C.).

The compound of formula (V) and its salt can be prepared from compound (VI), in which L¹ and A¹ are as described above, and compound (IV) (as described above). The reaction between compound (VI) and compound (IV) can readily occur in conditions described above.

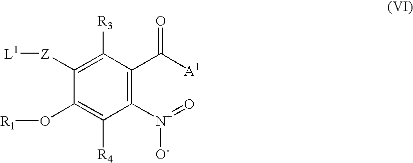

The present invention further aims to provide the application of the compounds mentioned above in production of medicaments for treating tumors.

The inventive tyrosine kinase inhibitor is a chemical compound capable of specifically acting on tyrosine kinase and inhibiting its activity, thereby inhibiting the activities of the two high-affinity receptors of VEGF and regulating the secretion of VEGF. VEGF is a major angiogenesis factor in tumor vascular tissue, and tumor VEGF expression is closely related to some of the complications of malignant solid tumors. Preclinical studies show that the tolerated doses of the tyrosine kinase inhibitor display good antitumor effect for animal model in animal test. Inhibition of VEGF secretion can indirectly inhibit tumor growth and achieve goals of treatment. The inventive therapy has the advantages of good targeting ability and low adverse side effect when compared with conventional treatment methods.

Receptor tyrosine kinases (RTKs) are proved to be important intracellular regulators of signal transduction. These proteins comprise an extracellular ligand binding site, transmembrane domain, and intracellular tyrosine kinases site. RTKs form dimer after binding with ligand and the dimer is thus activated. These enzymes can catalyze the transfer of phosphate group from ATP to tyrosine of the receptor tyrosine kinase. The specific inhibition related with KDR is utilized to inhibit the regulating signal of VEGF in the endothelial cell. Continuous angiogenesis results in tumor growth, and angiogenesis is the prerequisite of growth of solid tumor and formation of metastatic tumor. VEGF plays a critical role in the mentioned processes. The inventive compound is a protein inhibitor of KDR tyrosine kinase. Inhibition of VEGF can inhibit the angiogenesis driven by VEGF, which can be used to inhibit tumor growth and treat tumor.

Without further elaboration, it is believed that the above description has adequately enabled the present invention. The following specific embodiments are, therefore, to be construed as merely illustrative, and not limitative of the remainder of the disclosure in any way whatsoever. All of the publications cited herein are hereby incorporated by reference in their entirety.

### EXAMPLE 1

Compound 1 was synthesized from compound 2 and compound 3.

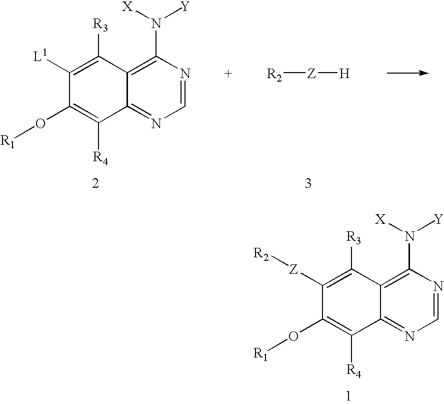

In the above compounds, X is —H, Y is methylphenyl, Z is

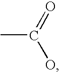

R₁ is methyl, R₂ is 4-ethylpiperidyl, R₃ is methyl, R₄ is —H, and L¹ is —Br.

Compounds 2 and 3 were added at equal molar amounts into 0.3 mol/L 2,6-dimethylpyridine solution containing 10% (v/v) of 0.1 mol Lethyl acetate. The reaction mixture was stirred and heated in water bath of 70° C. for 30 min. Then, it was filtered to collect the precipitate, which was dried to afford compound 1 as a white powder with water solubility and pH of 6.4.

### EXAMPLE 2

Compound 4, having formula (I) was synthesized from compound 5, having formula (III) and compound 6, having formula (IV).

In the above compounds, R₁ is ethyl, R₂ is 4-vinylpiperidyl, R₃ is —H, R₄ is methyl, X is methyl, Y is ethylphenyl, Z is

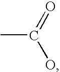

and L¹ is methoxyl.

Compounds 4 and 5 were added at equal molar amounts into 0.2 mol/L NaOH solution containing 8% (v/v) of 0.05 mol/L trichloromethane. The reaction mixture was stirred and heated in water bath of 50° C. for 30 min. Then, it was filtered to collect the precipitate, which was dried to afford compound 4 as a white powder with water solubility and pH of 6.4.

### EXAMPLE 3

Compound 7, having formula (I) was synthesized from compound 8, having formula (III) and compound 9, having formula (IV).

In the above compounds, R₁ is methyl, R₂ is 4-ethynylpiperidyl, R₃ is ethyl, R₄ is —H, X is methyl, Y is methylphenyl, Z is —NH, and L¹ is methoxyl.

Compounds 8 and 9 were added at equal molar amounts into 0.1 mol/L potassium carbonate solution containing 15% (v/v) of 0.05 mol/L isopropanol. The reaction mixture was stirred and heated in water bath of 100° C. for 20 min. Then, it was filtered to collect the precipitate, which was dried to afford compound 4 as a white powder with water solubility and pH of 6.4.

### EXAMPLE 4

Compound 10, having formula (I) was synthesized from compound 11, having formula (III) and compound 12, having formula (IV).

In the above compounds, R₁ is butyl, R₂ is 4-vinylpiperidyl, R₃ is pentynyl, R₄ is propenyl, X is propyl, Y is nitro, Z is —NH, and L¹ is phenoxy. Compounds 11 and 12 were added at equal molar amounts into 0.15 mol/L trimethylpyridine solution containing 12% (v/v) of 0.08 mol/L toluene. The reaction mixture was stirred and heated in water bath of 10° C. for 60 min. Then, it was filtered to collect the precipitate, which was dried to afford compound 4 as a white powder with water solubility and pH of 6.4.

### EXAMPLE 5

Compound 13, having formula (I) was synthesized from compound 14, having formula (III) and compound 15, having formula (IV).

In the above compounds, R₁ is propyl, R₂ is 4-vinylpiperidyl, R₃ is —H, R₄ is methyl, X is methyl, Y is ethylphenyl, Z is —S, and L¹ is methylsulfonoxy.

Compounds 14 and 15 were added at equal molar amounts into 0.08 mol/L 2,6-dimethylpyridine solution containing 6% (v/v) of 0.05 mol/L tetrahydrofuran. The reaction mixture was stirred and heated in water bath of 150° C. for 10 min. Then, it was filtered to collect the precipitate, which was dried to afford compound 4 as a white powder with water solubility and pH of 6.4.

### EXAMPLE 6

Compound 16, having formula (I) was synthesized from compound 17, having formula (III) and compound 18, having formula (IV).

In the above compounds, R₁ is ethyl, R₂ is 4-vinylpiperidyl, R₃ is butenyl, R₄ is methyl, X is methyl, Y is ethylphenyl, Z is

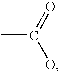

and L¹ is benzyl-4-sulfonoxy.

Compounds 17 and 18 were added at equal molar amounds into 0.2 mol/L sodium amide solution containing 5% (v/v) of 0.15 mol/L N,N-dimethylacetamide. The reaction mixture was stirred and heated in water bath of 120° C. for 30 min. Then, it was filtered to collect the precipitate, which was dried to afford compound 4 as a white powder with water solubility and pH of 6.4.

### EXAMPLE 7

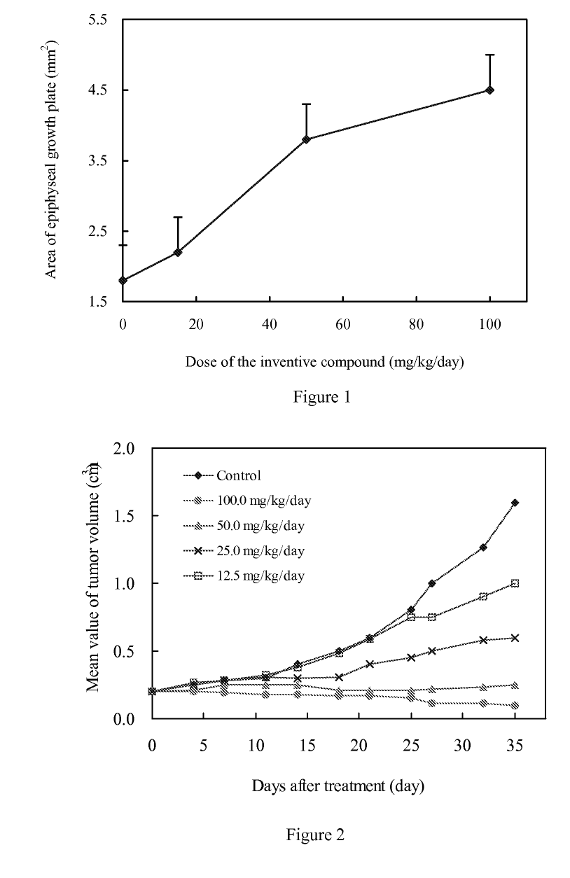

Compound 1 was administered continuously for 14 days to an Alderley Park young female rat (4-8 weeks old, wostar-derived) by subcutaneous injection at the dose of 0.25 mg/kg/d. The epiphyseal tissue structures of the leg joint of the rat was stained with hematoxylin and eosin. The binding site of the epiphyseal growth plate was measured for dose effect analysis. The results, shown in FIG. 1, indicate that the excess growth of the joint epiphyseal growth plate of rats resulted in growing dose dependence of the cartilage zone. When the dose was increased to 50 or 100 mg/kg/d, the inhibition on VEGF signal by the compound of the invention remained consistent with the in-vivo anti-angiogenesis effect.

### EXAMPLE 8

PC-3 human prostate tumors were implanted into nude mice (male, 6 weeks old). When the tumor volume reached 0.2 cm³, the mice were randomly divided into 5 groups, including a control group, a dose group of 100 mg/kg/d, a dose group of 50 mg/kg/d, a dose group of 25 mg/kg/d, and a dose group of 12.5 mg/kg/d. The test compound was administered to mice by intratumoral injection continuously for 7 days, and observed for 5 weeks. The results are shown in FIG. 2.

### EXAMPLE 9

Various tumors were inoculated at different body parts of 6-week old, male nude mice. The test compound was orally administered to the mice at different time points after the inoculation. The tumor weights were measured. Results are shown in table 1.

TABLE 1
Inhibition on human tumor transplanted into nude mice by the inventive compound
(orally administered)

| | | | | Days | | Inhibition | |
|---|---|---|---|---|---|---|---|
| | | | | after | | rate | |
| Transplanted | Tumor | Oral dosage | Test | transplantation | Administration | of tumor | P- |
| tumor | site | (mg/kg/day) | times | (d) | times | (%) | value |
| MDA-mb- | chest | 100 | 1 | 16 | 25 | 99 | <0.001 |
| 231 | | 50 | 1 | 16 | 25 | 82 | <0.001 |
| | | 25 | 1 | 16 | 25 | 64 | <0.01 |
| | | 12.5 | 1 | 16 | 25 | 71 | <0.001 |
| SKOV-3 | ovary | 100 | 1 | 18 | 28 | 100 | <0.001 |
| | | 50 | 1 | 18 | 28 | 98 | <0.001 |
| | | 25 | 1 | 18 | 28 | 50 | NS |
| | | 12.5 | 1 | 18 | 28 | 30 | NS |
| LoVo | colon | 100 | 2 | 5 | 14-17 | 99->100 | <0.001 |
| | | 50 | 2 | 5 | 14-17 | 77-81 | <0.01-0.001 |
| | | 25 | 2 | 5 | 14-17 | 55-60 | <0.05-0.001 |
| | | 12.5 | 2 | 5 | 14-17 | 5-27 | NS |
| A549 | lung | 100 | 1 | 14 | 25 | >100 | <0.001 |
| | | 50 | 1 | 14 | 25 | >100 | <0.001 |
| | | 25 | 1 | 14 | 25 | 88 | <0.001 |
| | | 12.5 | 1 | 14 | 25 | 64 | <0.001 |
| | | 12.5 | 1 | 14 | 21-30 | 15-46 | NS-<0.05 |
| A431 | pudendum | 100 | 1 | 14 | 21 | >100 | <0.001 |
| | | 50 | 2 | 14 | 21-30 | 83->100 | <0.001 |
| | | 25 | 2 | 14 | 21-30 | 42-80 | <0.05-<0.001 |
| | | 12.5 | 2 | 14 | 21-30 | 15-46 | NS-<0.05 |

NS = not significant

### EXAMPLE 10

Thymidine labeled by ³H (10 μCi/mL) and HUVEC (1×10⁵/mL) were co-cultured to allow integration of thymidine into HUVEC. A series of 10-fold dilutions of compound 1 in sterile distilled water were prepared from an initial concentration of 800 mg/L. The dilutions were separately added to into thymidine intergrated HUVEC. The cell division of HUVEC was measured after incubation, and IC₅₀ values of the compound for HUVEC were calculated.

As shown in Table 2, the compound significantly and selectively inhibited HUVEC proliferation induced by VEGF, and had no influence on growth of basal endothelial cell even at a concentration 50 times the IC₅₀ for the HUVEC proliferation induced by VEGF. Enzyme analysis shows that the compound exhibited different inhibitory activities against KDR, EGFR and FGFR1 (KDR>EGFR>FGFR1), and cellular composition analysis shows that the compound exhibited different inhibitory activities against VEGF, EGF and bFGF (VEGF>EGF>bFGF). Both analyses suggest that the compound have selective inhibitory activities against various growth factors.

TABLE 2
Inhibition of cell division of basal endothelial cell and growth
factor-induced HUVEC proliferation by the inventive compound

| | mean ± error | | | |
|---|---|---|---|---|
| | VEGF | EGF | FGF | Basal |
| IC₅₀(MM) | 0.06 ± 0.02 | 0.16 ± 0.03 | 0.8 ± 0.06 | >3 |
| Experiment | 6 | 6 | 5 | 4 |
| time | | | | |

EGF: epidermal growth factor
FGF: fibroblast growth factor
VEGF: vascular endothelial growth factor

### EXAMPLE 11

³H-containing thymine nucleotide (10 μCi/mL) and tumor cells were co-cultured to allow integration of thymidine into the cells. A series of dilutions of the test compound at a concentration gradient of 10⁻¹ starting from an initial concentration of 800 mg/L) were prepared. The solutions were added to the tumor cells integrated with the ³H-containing thymine nucleotide. The IC₅₀ of the test compound on tumor cells was calculated.

As shown in table 3, the inhibition range of the tumor cell growth IC₅₀ by the test compound was 0.8-1.4 mm, 13-230 times the concentration for inhibition of HUVEC division induced by VEGF. These data indicate that the tumor-resisting function of the compound in vivo is mainly to inhibit the endothelial VEGF signal factor, rather than to directly inhibit the growth of tumor cells.

TABLE 3
Influence of the inventive compound on tumor cell
division in vitro (n = 3)

| Tumor cell | | |
|---|---|---|
| line | Origin | Mean (±error) IC₅₀ (MM) |
| Calu-6 | lung | 1.30 ± 0.05 |
| MDA- | Chest | 6.00 ± 1.60 |
| MB-231 | | |
| SKOV-3 | ovary | 5.60 ± 0.10 |
| A431 | pudendum | 4.80 ± 0.10 |
| A549 | lung | 3.80 ± 0.40 |
| PC-3 | prostate | 3.70 ± 1.40 |
| LoVo | colon | 0.75 ± 0.20 |

Colon carcinoma LoVo cells were cultured in the logarithmic phase in a 96-well culture plate for 48 h. Compound (I) in sterile distilled water at a concentration of 0-100 μg/mL was added to the lovo cells (6 wells for each concentration). RPMI 1640 culture medium without the compound was used in the control group and the RPMI culture medium without the compound and cells was used in the blank control group. After 72 h, 20 μL MTT (5 g/L) was added into each well. After the culture plate was incubated at 37° C. for 4 h, the culture fluid was removed, 150 μL dimethyl sulfoxide (DMSO) was added to each well, and the absorbance (A) was measured at the wavelength of 570 nm.

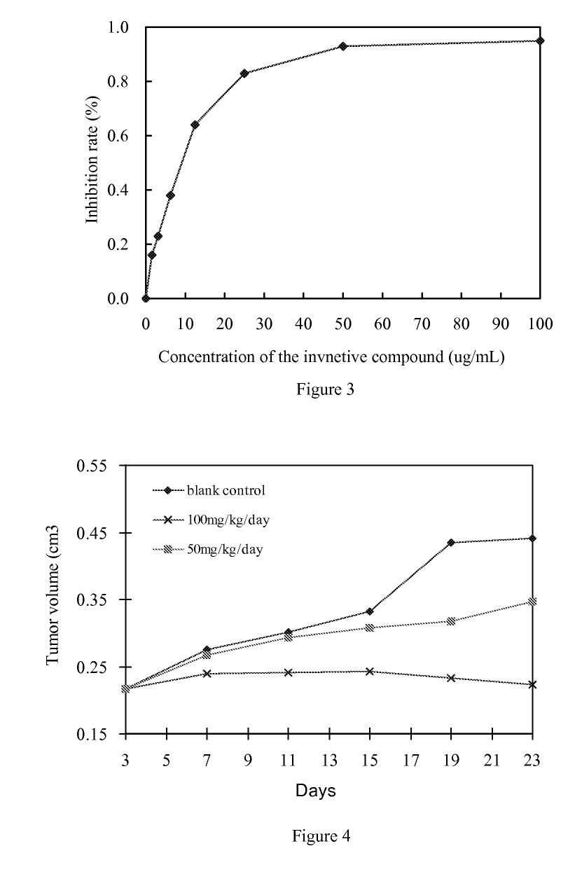

FIG. 3 show that the compound exhibited inhibitory effect on growth of Lovo cells in a dose-dependent manner. The inhibition rate was 50% when the concentration of the compound is 12.5 μg/mL, and over 90% when the concentration of the compound was 25 μg/mL.

### EXAMPLE 13

Compound (I) was intraperitoneally injected to a nude mice transplanted with colon carcinoma cell LoVo for 30 days at a dose of 100 mg/kg/day or 50 mg/kg/day. For blank control, 0.2 mL of 0.5% DMSO was intraperitoneally injected to nude mice. The mice were then sacrificed. The tumor volumes were measured for calculating T/C ratio, and weighing tumor for calculating inhibition rate (FIG. 4). The compound selectively inhibited the phosphorylation of KDR tyrosine kinase and blocked the signal transduction of tyrosine kinase, thereby inhibiting the tumor angiogenesis and tumor growth.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. For example, compounds structurally analogous to the quinazoline compounds of this invention also can be made, screened for their anti-tumor activities, and used to practice this invention. Thus, other embodiments are also within the claims.

What is claimed is:

1. A method of preparing a compound of formula (I): 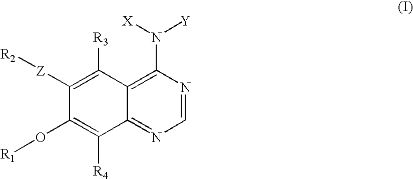
wherein
X represents H, methyl, or C₁₋₄ alkyl;
Y represents 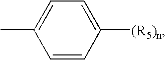
wherein n is 1, 2, 3, or 4, and R₅, being the same or different groups, is H, methyl, trifluoromethyl, nitro, cyano, C₂-C₄ alkyl, C₂-C₄ alkoxyl, N—(C₂-C₄)alkylamino, enzyme, hydroxyl, NN-triaza(C₁-C₄) alkylamino, C₂-C₄ alkylthio or C₂-C₄ alkylsulfonyl;
Z is 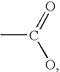
—O, —S or —NH;
R₁ represents methyl or C₁-C₄ alkyl;
R₂ represents C₁-C₅ alkyl-R₆, C₂-C₆ alkenyl-R₆, or C₂-C₆ alkynyl-R₆, in which the alkyl, alkenyl, and alkynyl are optionally substituted with one or more alkynyl, enzyme or amino; and R₆ is piperidin-4-yl, optionally substituted with one or more substituents of alkynyl, enzyme or amino;
R₃ and R₄ represent H, methyl, C₁-C₄ alkyl, C₂-C₆ alkenyl, C₂-C₆ alkynyl, cycloalkyl, or heterocycloalkyl;
the method comprising reacting a compound of formula (III): 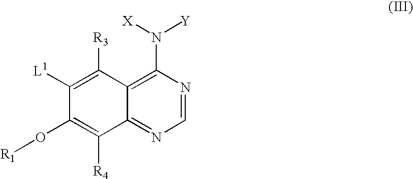
wherein L¹ represents halogen, alkoxyl, aryloxy, or sulfonoxy;
R₁ represents methyl or C₁-C₄ alkyl;
R₃ and R₄, independently, represent H, C₂-C₄ alkyl, C₂-C₆ alkenyl, C₂-C₆ alkynyl, cycloalkyl, or isocycloalkyl;
X represents H, methyl or C₁-C₄ alkyl; and
Y represents 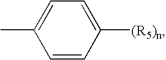
wherein n is 1, 2, 3, or 4, and R₅, being the same or different groups, is H, methyl, trifluoromethyl, nitro, cyano, C₂-C₄ alkyl, C₂-C₄ alkoxyl, N-(C₂-C₄)alkylamine, enzyme, hydroxyl, NN-triaza(C₁-C₄) alkylamino, C₂-C₄ alkylthio or C₂-C₄ alkylsulfonyl;
with a compound of formula (IV):
R₂-Z-H (IV)
wherein R₂ represents C₁-C₅ alkyl-R₆, C₂-C₆ alkenyl-R₆, C₂-C₆ alkynyl-R₆, in which the alkyl, alkenyl, and alkynyl are optionally substituted with one or more alkynyl, enzyme or amino; and R₆ is piperidin-4-yl, optionally substituted with one or more substituents of alkynyl, enzyme or amino; and
Z represents —O, —NH, 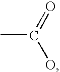
or —S.

2. The method of claim 1, wherein in formulas (III) and (IV), wherein
L¹ represents halogen or C1-C4 alkoxyl;
R₁ represents methyl;
R₂ represents C₁-C₅ alkyl-R₆;
R₃ represents C₁-C₄ alkyl;
R₄ represents H;
X represents H;
R₅ is alkyl; and
Z preferably represents

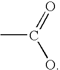

3. The method of claim 1, wherein in formulas (III) and (IV), wherein
L¹ is —Br or methoxyl;
R₁ is methyl;
R₂ is 4-ethylpiperidyl;
R₃ is methyl;
R₄ is —H;
X is H;
Y is methylphenyl; and
Z is

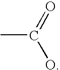

4. The method of claim 1, wherein the reaction is performed in the presence of base, in which the base is organic amine, carbonate or hydroxide of alkali metal or alkaline earth metal, alkali hydride, amide of alkali metal or alkaline earth metal.

5. The method of claim 4, wherein the base is 2,6-dimethylpyridine, trimethylpyridine, 4-dimethylaminopyridine, triethylamine, morphine, N-methylmorphine, 1,8-diazabicyclo(5,4,0)undecene-7, sodium carbonate, potassium carbonate, calcium carbonate, sodium hydroxide, potassium hydroxide, sodium hydride, sodium amide, or sodium bis(trimethylsilyl)amide.

6. The method of claim 5, wherein the base is 2,6-dimethylpyridine.

7. The method of claim 1, wherein the reaction is performed in the presence of an inert solvent.

8. The method of claim 1, wherein the inert solvent is selected from methanol, ethanol, isopropanol, ethyl acetate, dichloromethane, trichloromethane, carbon tetrachloride, tetrahydrofuran, 1,4-dioxane, toluene, N,N-dimethylformamide, N,N-dimethylacetamide, N-methylpyrrol-2-one, and dimethyl sulfoxide.

9. The method of claim 8, wherein the inert solvent is ethyl acetate.

10. The method of claim 1, wherein the reaction is performed at a temperature of 10-150° C.

11. The method of claim 10, wherein the temperature is 100° C.

12. The method of claim 2, wherein the reaction is performed in the presence of base, in which the base is organic amine, carbonate or hydroxide of alkali metal or alkaline earth metal, alkali hydride, amide of alkali metal or alkaline earth metal.

13. The method of claim 12, wherein the base is 2,6-dimethylpyridine, trimethylpyridine, 4-dimethylaminopyridine, triethylamine, morphine, N-methylmorphine, 1,8-diazabicyclo(5,4,0)undecene-7, sodium carbonate, potassium carbonate, calcium carbonate, sodium hydroxide, potassium hydroxide, sodium hydride, sodium amide, or sodium bis(trimethylsilyl)amide.

14. The method of claim 13, wherein the base is 2,6-dimethylpyridine.

15. The method of claim 2, wherein the reaction is performed in the presence of an inert solvent.

16. The method of claim 15, wherein the inert solvent is selected from methanol, ethanol, isopropanol, ethyl acetate, dichloromethane, trichloromethane, carbon tetrachloride, tetrahydrofuran, 1,4-dioxane, toluene, N,N-dimethylformamide, N,N-dimethylacetamide, N-methylpyrrol-2-one, and dimethyl sulfoxide.

17. The method of claim 16, wherein the inert solvent is ethyl acetate.

18. The method of claim 2, wherein the reaction is performed at a temperature of 10-150° C.

19. The method of claim 18, wherein the temperature is 100° C.
